(19) United States
(12) Patent Application Publication
(10) Pub. No.: US 2023/0293611 A1
(43) Pub. Date: Sep. 21, 2023

(54) NATURAL PRODUCT EXTRACTS AND METHODS OF USE THEREOF

(71) Applicant: THE ADORED BEAST APOTHECARY LTD., Toronto (CA)

(72) Inventors: Julie LEE, Toronto (CA); H.P. Vasantha RUPASINGHE, Bible Hill (CA)

(21) Appl. No.: 18/041,476

(22) PCT Filed: Aug. 13, 2021
(86) PCT No.: PCT/CA2021/051117

§ 371 Date: Feb. 13, 2023

Related U.S. Application Data
(60) Provisional application No. 63/065,021, filed on Aug. 13, 2020.

(51) Int. Cl. A61K 36/07; A61K 36/05; A61P 35/00

(52) U.S. Cl. CPC A61K 36/07; A61K 36/05; A61P 35/00; A61K 2236/333; A61K 2236/53

(57) ABSTRACT

Described herein are various natural product extracts. For example, an alcohol extract of chaga is described herein. A substantially chlorophyll-free extract of phytoplankton green algae is also described herein. The extracts may be used alone or in combinations with each other and/or other active agents for preventing and/or treating inflammation, for preventing and/or treating oxidation, stimulating the immune system, for preventing and/or treating cancer, and/or for preferentially killing cancer cells over non-cancerous cells.

### FIELD

The present invention relates to extracts. More specifically, the present invention is, in embodiments, concerned with extracts of natural products and related products, methods, and uses.

### BACKGROUND

Inonotus obliquus, commonly known as chaga, is a fungus in the family Hymenochaetaceae. It is parasitic on birch and other trees. Chaga is traditionally grated into a fine powder and used to brew a beverage resembling coffee or tea.

The green algae are a highly paraphyletic group within the green plants (Viridiplantae) and includes at least 7,000 species of mostly aquatic photosynthetic eukaryotic organisms. Some species of algae, particularly chlorophyll-containing green algae, have been used as health supplements

There is a need for natural health products that are useful in the treatment and/or prevention of human or animal health conditions and/or as natural health supplements.

### DESCRIPTION OF THE DRAWINGS

The present invention will be further understood from the following description with reference to the Figures, in which:

FIG. 1 shows the inhibitory effects of various chaga extracts on (a) Hepg2 and (b) MDA-MB-231 cells.

FIG. 2 shows the inhibitory effects of various green algae extracts on (a) Hepg2 and (b) MDA-MB-231 cells.

FIG. 3 shows morphology alterations in (a) HepG2 and (b) MDA-MB-231 cells treated with vehicle, control drugs (sorafenib or doxorubicin), or methanol extract of chaga for 24 h.

FIG. 4 shows the inhibitory effects of (a) methanol and (b) water extracts of chaga on MDA-MB-231 cells at 24 and 48 h.

FIG. 5 shows the inhibitory effects of methanol extract of chaga on HepG2 cells at 24 and 48 h.

FIG. 6 shows (A) histograms; M1: live cells and M2: dead cells and (B) bar graphs (derived from A) showing % dead cells.

FIG. 7 shows a dose dependent effect of chaga on D17 osteosarcoma cells and WRL68 hepatocytes, as measured in an MTS assay. In FIG. 7A, the chaga was extracted with methanol. In FIG. 7B, the chaga was extracted with water.

FIG. 8 shows a dose dependent effect of chaga on D17 osteosarcoma cells and WRL68 hepatocytes, as measured in an ACP assay. In FIG. 8A, the chaga was extracted with methanol. In FIG. 8B, the chaga was extracted with water.

FIG. 9 shows a dose dependent effect of a methanol extract of chaga on D17 osteosarcoma cells. FIG. 9A is a control; FIG. 9B is 400 μg/ml; FIG. 9C is 600 μg/ml.

FIG. 10 shows different fractions of phytoplankton green algae. FIG. 10A shows the top chlorophyll-containing organic phase and FIG. 10B shows the bottom organic phase, which is substantially free of chlorophyll.

FIG. 11 shows a dose dependent effect of phytoplankton green algae on D17 osteosarcoma cells and WRL68 hepatocytes, as measured in an MTS assay. In FIG. 11A, the phytoplankton green algae fraction contained chlorophyll. In FIG. 11B, the phytoplankton green algae fraction was substantially free of chlorophyll.

FIG. 12 shows a dose dependent effect of phytoplankton green algae on D17 osteosarcoma cells and WRL68 hepatocytes, as measured in an ACP assay. In FIG. 12A, the phytoplankton green algae fraction contained chlorophyll. In FIG. 12B, the phytoplankton green algae fraction was substantially free of chlorophyll.

FIG. 13 shows a dose dependent effect of a substantially chlorophyll free extract of phytoplankton green algae on D17 osteosarcoma cells. FIG. 13A is a control; FIG. 13B is 400 μg/ml; FIG. 13C is 600 μg/ml.

FIG. 14 shows inhibitory effects of MH-E1-SF, MH-SFE1b, MH-SFE1c, GLE1-CF and GLE1-CF-SF in human and canine cancer cell lines. One-way anova analysis was used to compare the dose dependent toxicity. *p<0.05, **p<0.01, ***p<0.001 and ****p<0.0001 compared with control group.

FIG. 15 shows morphological assessments of D-17 cell under phase-contrast microscope (100×, Nikon). A. Representative morphological images of D-17 canine osteosarcoma cells in various concentrations of GLE1-CF-SF and B. MH-E1-SF extracts after 24 h treatment.

FIG. 16 shows drug dose-response curves. The combination of MH-E1-SF and GLE1-CF-SF (1:1) enhances cellular toxicity significantly in MCF-7, HepG2, D-17, and DH-82 cell lines. Two-way ANOVA analysis was performed. * compares with MH-E1-SF and #compares with GL-E1-SF.

FIG. 17 shows combination effect and isobologram analysis. The combination of GL (GL-E1-SF) and MH (MH-E1-SF) in 1:1, 1:2, and 1:4 were quantified by analyzing isoboles. 1:1 combination results in synergy effect in HepG2 cells and additive effect in other cell lines. 1:2 and 1:4 combination of GL and MH results in synergy effect in HepG2, HOS, D-17, DH-82 except for MCF-7 cells (additive effect).

### SUMMARY

In accordance with an aspect, there is provided an alcohol extract of chaga.

In an aspect, the alcohol extract is substantially carbohydrate-free.

In an aspect, the alcohol comprises a lower alcohol, such as methanol, ethanol, propanol, isopropanol, butanol, sec-butyl alcohol, isobutyl alcohol, t-butyl alcohol, or a combination thereof.

In an aspect, the alcohol extract is collected from a supernatant of chaga dissolved in alcohol.

In an aspect, the extract has anti-inflammatory effects.

In an aspect, the extract has anti-oxidative effects.

In an aspect, the extract stimulates the immune system.

In an aspect, the extract has anticancer effects.

In an aspect, the extract is preferentially cytotoxic to cancer cells as compared to non-cancerous cells.

In an aspect, the extract is further for preventing and/or treating inflammation.

In an aspect, the extract is further for preventing and/or treating oxidation.

In an aspect, the extract is further for stimulating the immune system.

In an aspect, the extract is for preventing and/or treating cancer.

In an aspect, the extract is for preferentially killing cancer cells over non-cancerous cells.

In an aspect, the extract is for use in a veterinary animal such as a dog.

In accordance with an aspect, there is provided a substantially chlorophyll-free extract of phytoplankton green algae.

In an aspect, the substantially chlorophyll-free extract is substantially chlorophyll-free.

In an aspect, the phytoplankton green algae comprises microalgae.

In an aspect, the microalgae comprises Spirulina,

Chlorella, Tetraselmis, Nannochloropsis, Nitzchia, Navicula, Scenedesmus, Crypthecodinium, Chaetoceros, or combinations thereof.

In an aspect, the microalgae comprises Nannochloropsis, such as Nannochloropsis gaditana, Tetraselmis, such as Tetraselmis chui, Chlorella, such as Chlorella vulgaris, or combinations thereof.

In an aspect, the microalgae comprises Nannochloropsis gaditana, Tetraselmis chui, and Chlorella vulgaris.

In an aspect, the microalgae comprises 60% w/w Nannochloropsis gaditana, 15% w/w Tetraselmis chui, and 25% w/w Chlorella vulgaris.

In an aspect, the substantially chlorophyll-free extract is collected from an aqueous phase of phytoplankton green algae dissolved in a 2-phase solvent.

In an aspect, the 2-phase solvent comprises n-heptane, ethanol, acetonitrile and water. In an aspect, the n-heptane, ethanol, acetonitrile and water are in a ratio of 10:8:1:1, vol/vol, respectively.

In an aspect, the extract comprises less than about 5%, 4%, 3%, 2% 1%, 0.5%, or 0.1% w/v chlorophyll.

In an aspect, the extract has anti-inflammatory effects.

In an aspect, the extract has anti-oxidative effects.

In an aspect, the extract stimulates the immune system.

In an aspect, the extract has anticancer effects.

In an aspect, the extract is preferentially cytotoxic to cancer cells as compared to non-cancerous cells.

In an aspect, the extract is for preventing and/or treating inflammation.

In an aspect, the extract is for preventing and/or treating oxidation.

In an aspect, the extract is for stimulating the immune system.

In an aspect, the extract is for preventing and/or treating cancer.

In an aspect, the extract is for preferentially killing cancer cells over non-cancerous cells.

In an aspect, the extract is for use in a veterinary animal such as a dog.

In accordance with an aspect, there is provided a combination comprising the alcohol extract of chaga described herein and the substantially chlorophyll-free extract of phytoplankton green algae described herein.

In an aspect, the alcohol extract of chaga and the substantially chlorophyll-free extract of phytoplankton green algae are in synergistic amounts.

In an aspect, the synergistic amounts comprise a ratio of from about 1:10 to about 10:1 of the alcohol extract of chaga to the substantially chlorophyll-free extract of phytoplankton green algae.

In an aspect, the synergistic amounts comprise a ratio of from about 1:4 to about 4:1 of the alcohol extract of chaga to the substantially chlorophyll-free extract of phytoplankton green algae.

In an aspect, the synergistic amounts comprise a ratio of 1:4 or 4:1 of the alcohol extract of chaga to the substantially chlorophyll-free extract of phytoplankton green algae.

In accordance with an aspect, there is provided a composition comprising the combination described herein.

In accordance with an aspect, there is provided a kit comprising the combination described herein.

In accordance with an aspect, there is provided a method for preventing and/or treating inflammation, the method comprising administering the methanol extract; the substantially chlorophyll-free extract; or the combination described herein to a subject in need thereof.

In accordance with an aspect, there is provided a method for preventing and/or treating oxidation, the method comprising administering the methanol extract; the substantially chlorophyll-free extract; or the combination described herein to a subject in need thereof.

In accordance with an aspect, there is provided a method for stimulating the immune system, the method comprising administering the methanol extract; the substantially chlorophyll-free extract; or the combination described herein to a subject in need thereof.

In accordance with an aspect, there is provided a method for preventing and/or treating cancer, the method comprising administering the methanol extract; the substantially chlorophyll-free extract; or the combination described herein to a subject in need thereof.

In accordance with an aspect, there is provided a method for preferentially killing cancer cells over non-cancerous cells, the method comprising administering the methanol extract; the substantially chlorophyll-free extract; or the combination described herein to a subject in need thereof.

In an aspect, the subject is a mammal.

In an aspect, the mammal is a veterinary animal, such as a dog.

In accordance with an aspect, there is provided a use of the methanol extract; the substantially chlorophyll-free extract; or the combination described herein for preventing and/or treating inflammation in a subject.

In accordance with an aspect, there is provided a use of the methanol extract; the substantially chlorophyll-free extract; or the combination described herein for preventing and/or treating oxidation in a subject.

In accordance with an aspect, there is provided a use of the methanol extract; the substantially chlorophyll-free extract; or the combination described herein for stimulating the immune system in a subject.

In accordance with an aspect, there is provided a use of the methanol extract; the substantially chlorophyll-free extract; or the combination described herein for preventing and/or treating cancer in a subject.

In accordance with an aspect, there is provided a use of the methanol extract; the substantially chlorophyll-free extract; or the combination described herein for preferentially killing cancer cells over non-cancerous cells in a subject.

In an aspect, the subject is a mammal.

In an aspect, the mammal is a veterinary animal, such as a dog.

In accordance with an aspect, there is provided a method for producing an active chaga extract, the method comprising dissolving the chaga in alcohol and collecting a resulting supernatant.

In an aspect, the method further comprises sonicating the dissolved chaga before collecting the supernatant.

In an aspect, the method further comprises centrifuging the dissolved chaga before collecting the supernatant.

In an aspect, the method further comprises evaporating the supernatant to remove methanol.

In an aspect, the method further comprises dissolving the evaporated supernatant in a solvent.

In accordance with an aspect, there is provided a method for producing an active substantially chlorophyll-free phytoplankton green algae extract, the method comprising dissolving the phytoplankton green algae in a 2-phase solvent and collecting a resulting aqueous phase.

In an aspect, the 2-phase solvent comprises n-heptane, ethanol, acetonitrile and water.

In an aspect, the n-heptane, ethanol, acetonitrile and water are in a ratio of 10:8:1:1, vol/vol, respectively.

In an aspect, the method further comprises shaking and/or sonicating the dissolved phytoplankton green algae before collecting the collecting the aqueous phase.

In an aspect, the method further comprises equilibrating the dissolved phytoplankton green algae for a period of time before collecting the aqueous phase.

In an aspect, the method further comprises repeating the method by extracting the aqueous phase with the 2-phase solvent.

In an aspect, the method further comprises filtering the aqueous phase.

In an aspect, the method further comprises evaporating the aqueous phase.

In an aspect, the method further comprises dissolving the evaporated aqueous phase in a solvent.

In an aspect, the extract comprises less than about 5%, 4%, 3%, 2%, 1%, 0.5%, or 0.1% w/v chlorophyll.

In accordance with an aspect, there is provided an extract made by the method described herein.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### DETAILED DESCRIPTION

Described herein are extracts of natural products such as chaga and phytoplankton green algae, which may be used individually or in combination. Also described are methods of extracting the natural products and various methods and uses for the products.

### Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning. A Laboratory Manual, Cold Springs Harbor Press (Cold Springs Harbor, N Y 1989), each of which are incorporated herein by reference. For the purposes of the present invention, the following terms are defined below.

The term “substantially free” herein means less than about 5%, typically less than about 2%, more typically less than about 1%, even more typically less than about 0.5%, most typically less than about 0.1% contamination, such as with chlorophyll and/or carbohydrates, such as sugar.

As used herein, “treatment” or “therapy” is an approach for obtaining beneficial or desired clinical results. For the purposes described herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” and “therapy” can also mean prolonging survival as compared to expected survival if not receiving treatment or therapy. Thus, “treatment” or “therapy” is an intervention performed with the intention of altering the pathology of a disorder. Specifically, the treatment or therapy may directly prevent, slow down or otherwise decrease the pathology of a disease or disorder such as inflammation, or may render the inflammation more susceptible to treatment or therapy by other therapeutic agents.

The terms “therapeutically effective amount”, “effective amount” or “sufficient amount” mean a quantity sufficient, when administered to a subject, including a mammal, for example a human, to achieve a desired result, for example an amount effective to treat and/or prevent inflammation and/or cancer. Effective amounts of the extracts described herein may vary according to factors such as the disease state, age, sex, and weight of the subject. Dosage or treatment regimes may be adjusted to provide the optimum therapeutic response, as is understood by a skilled person.

Likewise, an “effective amount” of the extracts described herein refers to an amount sufficient to function as desired, such as to treat and/or prevent inflammation and/or cancer.

The term “subject” as used herein refers to any member of the animal kingdom, including birds, fish, invertebrates, amphibians, mammals, and reptiles. Typically, the subject is a human or non-human vertebrate. Non-human vertebrates include livestock animals, companion animals, and laboratory animals. Non-human subjects also specifically include non-human primates as well as rodents. Non-human subjects also specifically include, without limitation, poultry, chickens, horses, cows, pigs, goats, dogs, cats, guinea pigs, hamsters, mink, rabbits, crustaceans, and molluscs. Typically the subject is poultry or a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. In typical aspects, the mammal is human or a pet animal such as a dog.

Administration “in combination with” one or more further therapeutic agents includes simultaneous (concurrent) and consecutive administration in any order. Combinations described herein may work additively or synergistically.

The term “pharmaceutically acceptable” means that the extract or combination of extracts is compatible with the remaining ingredients of a formulation for pharmaceutical use, and that it is generally safe for administering to humans according to established governmental standards, including those promulgated by the United States Food and Drug Administration.

“Carriers” as used herein include cosmetically or pharmaceutically acceptable carriers, excipients, or stabilizers that are nontoxic to the cell or subject being exposed thereto at the dosages and concentrations employed. Often the pharmaceutically acceptable carrier is an aqueous pH buffered solution. Examples of pharmacologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, and dextrins; chelating agents such as EDTA; sugar alcohols such as mannitol and sorbitol; salt-forming counterions such as sodium; and/or nonionic surfactants such as TWEEN™, polyethylene glycol (PEG), and PLURONICS™.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any embodiments described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation. For example, in embodiments, chlorophyll is explicitly excluded from the compositions and methods described herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Extracts

Described herein are various natural product extracts and purified fractions thereof. For example, an alcohol extract of chaga is described herein. The chaga may be extracted with any alcohol but, typically, a lower alcohol is used. Lower alcohols have four or fewer carbon atoms and include, for example, methanol, ethanol, propanol, isopropanol, butanol, sec-butyl alcohol, isobutyl alcohol, and t-butyl alcohol. Combinations of alcohols, including combinations of lower alcohols, are contemplated for use herein. The alcohol is typically collected from a supernatant of chaga dissolved in alcohol.

In other aspects, described herein is a substantially chlorophyll-free extract of phytoplankton green algae. Any species of phytoplankton green algae is contemplated for use herein, however, typically the phytoplankton green algae comprises microalgae. The microalgae typically comprises Spirulina, Chlorella, Tetraselmis, Nannochloropsis, Nitzchia, Navicula, Scenedesmus, Crypthecodinium, Chaetoceros, or combinations thereof. For example, Nannochloropsis gaditana, Tetraselmis chui, Chlorella vulgaris, or combinations thereof may be used. In particular aspects, the phytoplankton green algae may comprise various combinations of algae, including microalgae.

In aspects, the microalgae comprises Nannochloropsis gaditana, Tetraselmis chui, and Chlorella vulgaris. These species may be combined in any amounts and ratios. For example, from about 1 to about 99% w/w of each species. In typical aspects, the microalgae comprises about 60% w/w Nannochloropsis gaditana, about 15% w/w Tetraselmis chui, and about 25% w/w Chlorella vulgaris.

Typically, the substantially chlorophyll-free extract is collected from an aqueous phase of phytoplankton green algae dissolved in a 2-phase solvent. The 2-phase solvent typically comprises n-heptane, ethanol, acetonitrile and water, optionally in a ratio of 10:8:1:1, vol/vol, respectively.

By “substantially free” of chlorophyll, it is mean that the extract comprises less than about 5%, 4%, 3%, 2%, 1%, 0.5%, or 0.1% w/v chlorophyll or in some aspects the extract is free of chlorophyll. Similarly, the alcohol extracts of chaga and substantially chlorophyll-free extracts of phytoplankton green algae may be substantially free of carbohydrates such as sugar, meaning that the extract comprises less than about 5%, 4%, 3%, 2%, 1%, 0.5%, or 0.1% w/v carbohydrate or sugar or in some aspects the extract is free of carbohydrates or sugar.

It has been found that alcohol extracts of chaga and substantially chlorophyll-free extracts of phytoplankton green algae provide certain beneficial properties. For example, in aspects, the extracts have anti-inflammatory effects, anti-oxidative effects, and/or anticancer effects. In additional or alternative aspects, the extracts stimulate the immune system and/or are preferentially cytotoxic to cancer cells as compared to non-cancerous cells. In additional or alternative aspects, the extracts have anti-aging effects and/or support gut health and/or support a healthy microbiome.

Thus, it will be appreciated that, in aspects, the extracts may be used for preventing and/or treating inflammation, for preventing and/or treating oxidation, stimulating the immune system, for preventing and/or treating cancer, and/or for preferentially killing cancer cells over non-cancerous cells.

It will be understood that the extracts can be used in any member of the animal kingdom, including birds, fish, invertebrates, amphibians, mammals, and reptiles. Typically, the subject is a human or non-human vertebrate. Non-human vertebrates include livestock animals, companion animals, and laboratory animals. Non-human subjects also specifically include non-human primates as well as rodents. Non-human subjects also specifically include, without limitation, poultry, chickens, horses, cows, pigs, goats, dogs, cats, guinea pigs, hamsters, mink, rabbits, crustaceans, and molluscs. Typically the subject is a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is a dog.

In particular aspects, the alcohol extract of chaga and the substantially chlorophyll-free extract of phytoplankton green algae may be used in combination to provide additive or synergistic effects. The extracts may be used in any amounts or ratios, but are typically used in synergistic amounts for achieving one or more of the effects described herein. For example, the extracts may be used in ratios of from about 1:1000 to about 1000:1 chaga:phytoplankton green algae, such as from about 1:100 to about 100:1, such as from about 1:10 to about 10:1, such as from about 1:10, 1:9, 1:8, 1:7, 1:6, 1:5, 1:4, 1:3, 1:2, or 1:1 to about 1:1, 2:1, 3:1, 4:1, 5:1, 6:1, 7:1, 8:1, 9:1, or 10:1.

In aspects, the alcohol extract of chaga and/or the substantially chlorophyll-free extract of phytoplankton green algae may be administered to a subject, such as a mammal, such as a dog or human, in any suitable amount. For example, these may be administered in single or combined doses of from about 0.01 mg/kg to about 1000 mg/kg administered orally or parenterally, such as by IV. For example, from about 0.01, about 0.05, about 0.1, about 0.5, about 1, about 2, about 3, about 4, about 5, about 6, about 7, about 8, about 9, about 10, about 15, about 20, about 25, about 50, about 75, about 100, about 200, about 300, about 400, about 500, about 600, about 700, about 800, or about 900 mg/kg to about 0.05, about 0.1, about 0.5, about 1, about 2, about 3, about 4, about 5, about 6, about 7, about 8, about 9, about 10, about 15, about 20, about 25, about 50, about 75, about 100, about 200, about 300, about 400, about 500, about 600, about 700, about 800, about 900 or about 1000 mg/kg. For example, in some aspects, from about 1 mg/kg to about 50 mg/kg for oral doses or from about 0.1 mg/kg to about 5 mg/kg for IV doses.

The chaga and phytoplankton green algae extracts may be used together in a single composition or administered separately to the same subject simultaneously or sequentially, in any order, optionally provided in a kit.

The extracts may be administered over a period of hours, days, weeks, or months, depending on several factors, including the severity and type of the inflammation or other condition being treated, whether a recurrence is considered likely, or to prevent the inflammation or other condition, etc. The administration may be constant, e.g., constant infusion over a period of hours, days, weeks, months, etc. Alternatively, the administration may be intermittent, e.g., the extracts may be administered once a day over a period of days, once an hour over a period of hours, or any other such schedule as deemed suitable.

The compositions described herein can be prepared by per se known methods for the preparation of pharmaceutically or cosmetically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the entirety of which is incorporated herein by reference).

Pharmaceutically acceptable carriers are well known to those skilled in the art and include, for example, sterile saline, lactose, sucrose, calcium phosphate, gelatin, dextrin, agar, pectin, peanut oil, olive oil, sesame oil, cannabis oil, and water. Furthermore the composition may comprise one or more stabilizers such as, for example, carbohydrates including sorbitol, mannitol, starch, sucrose, dextrin and glucose, proteins such as albumin or casein, and buffers like alkaline phosphates.

The compositions described herein can, in embodiments, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, intravaginal, aerosol, oral, topical, or transdermal administration. Typically, the compositions of the invention are administered orally.

It is understood by one of skill in the art that the compositions described herein can be used in conjunction with known therapies for prevention and/or treatment of inflammation or cancer, for example, in subjects. Similarly, the compositions described herein can be combined with one or more other natural products or extracts thereof. The compositions described herein may, in embodiments, be administered in combination, concurrently or sequentially, with conventional treatments for inflammation, cancer, or other conditions, including non-steroidal anti-inflammatory drugs or chemotherapy, for example. The compositions described herein may be formulated together with such conventional treatments when appropriate.

Thus, also described herein are methods of use of the extracts described herein. For example, described herein are methods for preventing and/or treating inflammation, preventing and/or treating oxidation, stimulating the immune system, preventing and/or treating cancer, and/or preferentially killing cancer cells over non-cancerous cells. The method comprises administering the methanol extract; the substantially chlorophyll-free extract; or the combination described herein to a subject in need thereof.

Also described are methods for producing the extracts described herein. For example, an active chaga extract is typically made by dissolving the chaga in alcohol, optionally sonicating the dissolved chaga and centrifuging the resulting solution, and collecting the resulting supernatant. The supernatant is typically evaporated to remove alcohol, such as methanol or ethanol, which is then dissolved in a suitable solvent.

In other aspects, described herein is a method for producing an active substantially chlorophyll-free phytoplankton green algae extract. The method typically comprises dissolving the phytoplankton green algae in a 2-phase solvent, optionally shaking and/or sonicating the dissolved phytoplankton green algae, optionally equilibrating the dissolved phytoplankton green algae for a period of time, and collecting a resulting aqueous phase. The 2-phase solvent typically comprises n-heptane, ethanol, acetonitrile and water, which are typically in a ratio of 10:8:1:1, vol/vol, respectively. The extraction may be repeated one or more times. In aspects, the aqueous phase is filtered and evaporated and dissolved in a suitable solvent.

Also provided are extracts made by the methods described herein.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1: Screening of Cytotoxicity of Selected Natural Health Products

The aim of this project is to study the cytotoxic effects of Chaga mushroom Inonotus obliquus (MH) and phytoplankton green algae (GL) extracts on liver carcinoma HepG2 and breast carcinoma MDA-MB-231 cells.

### Methods

(a) Solvent extraction: Four different solvents were used, namely, methanol, hexane:ethyl acetate (39:1), ethyl acetate, and water. Briefly, 2 g of the test material (MH/GL) were dissolved in 80 ml of appropriate solvents (methanol/hexane:ethyl acetate/ethyl acetate), sonicated twice for 20 min at room temperature (RT), and centrifuged at 3000 rpm for 10 min. The supernatant was collected and evaporated using a rotary evaporation system at 65° C. The concentrated extract was subjected to nitrogen evaporation to remove all the solvent, then dissolved in DMSO to a final concentration of 60 mg/ml. For water extraction, 2 g of the test material was dissolved in 80 ml water, sonicated twice for 20 min at RT, kept at RT for three days, vortexed, and centrifuged at 3000 rpm for 10 min. The supernatant was then freeze-dried into powder. The powder was dissolved in DMSO to a final concentration of 60 mg/ml.
(b) MTS assay: The potential inhibitory effects of MH and GL extracts on HepG2 and MDA-MB-231 cells were measured using MTS assay. Briefly, 6000 cells were seeded and treated with different concentrations (6.25, 12.5, 25, 50, 100, 150, and 300 μg/ml) of extracts for 24 h. Then, MTS/PMS reagent was added and absorbance was measured at 490 nm using a plate reader. Sorafenib (20 μM) and doxorubicin (50 μM) were used as positive controls for HepG2 and MDA-MB-231 cells, respectively.

### Results

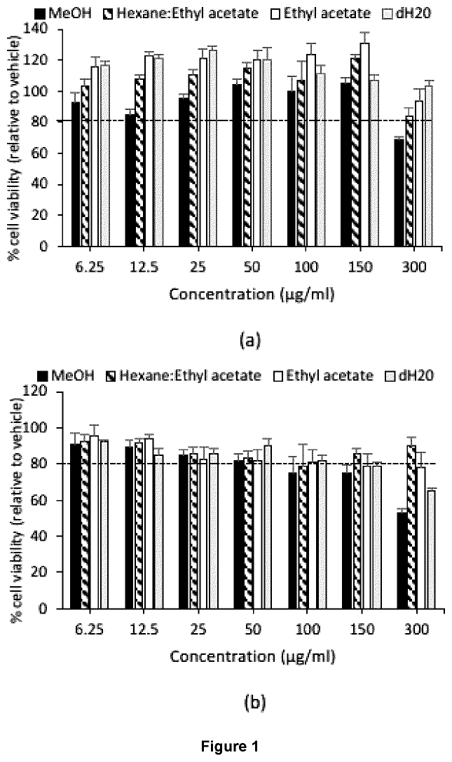

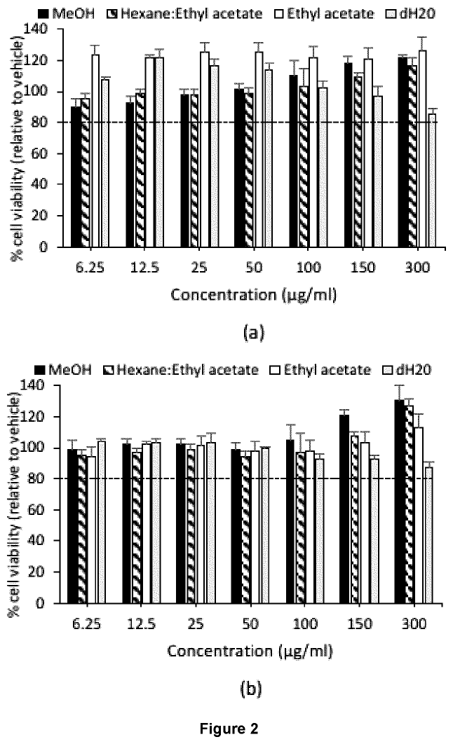

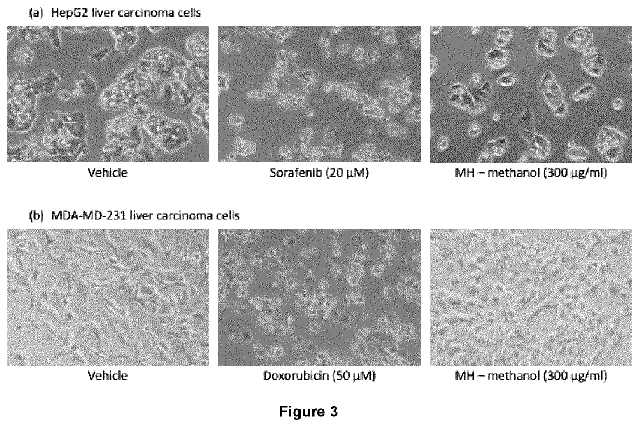

FIG. 1 and FIG. 2 show the inhibitory effects of various MH and GL extracts, respectively, on the viability of HepG2 and MDA-MB-231 cells. This study shows that certain MH extracts were markedly effective. At the highest treatment dose (300 μg/ml), methanol extract of MH markedly reduced the viability of HepG2 (69.1±1.8%) and MDA-MD-231 (53.0±5.4%) cells (FIGS. 1a and 1b). Water extract of MH also markedly reduced the viability of MDA-MB-231 cells to 64.7±2.0% (FIG. 1b). Both positive controls, sorafenib and doxorubicin, reduced HepG2 and MDA-MB-231 cell viability, respectively, to 26.0±3.7% and 24.4±5.1% (mean±SEM, n=3) following 24 h treatment (data not shown). FIG. 3 shows the representative photographs taken at 24 h post-treatment of vehicle, positive controls, and methanol extract of MH on HepG2 and MDA-MB-231 cells.

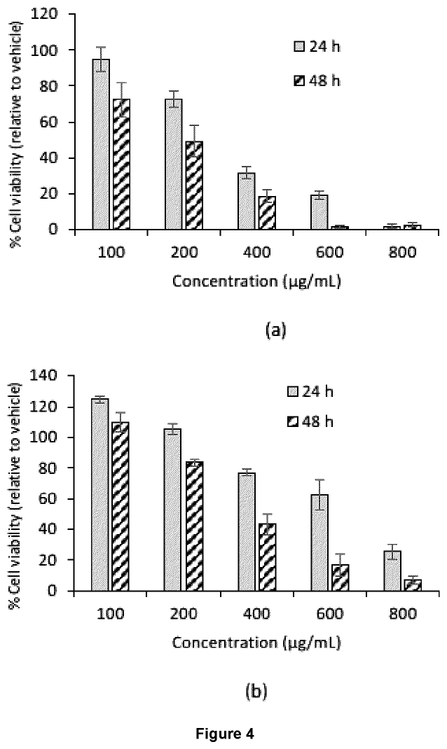

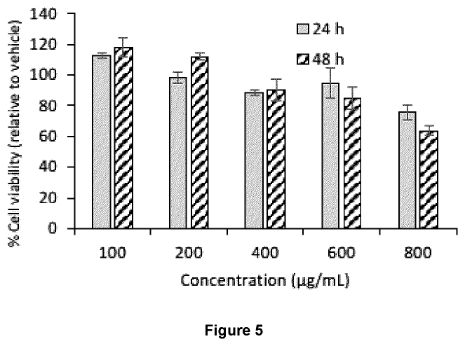

The MTS assay was repeated with 100, 200, 400, 600, 800 μg/ml of MH methanol extract for HepG2 and MDA-MB-231 cells; and 100, 200, 400, 600, 800 μg/ml of MH water extract for MDA-MB-231 cells at 24 h and 48 h (FIG. 4 and FIG. 5).

### Conclusion

The methanol extract of MH markedly reduced the viability of both HepG2 and MDA-MB-231 cells, whereas the water extract of MH markedly reduced the viability of MDA-MB-231 cells. The MTS results showed that high concentrations of methanol and water extracts of MH exert dose-dependent inhibitory effects on MDA-MB-231 cells at 24 and 48 h.

### Example 2: Confirmation of Inhibitory Effects of Chaga on Cancer Cells

### Method

MDA-MB-231 cells were treated with 200 and 400 μg/ml of methanol and water extracts for 48 h at 37° C. Cells were then harvested and washed with PBS. Cells were incubated with 0.25 μg of 7-aminoactinomycin D (7-AAD) viability staining solution at room temperature for 5 min and analyzed using a FACS Calibur flow cytometer.

### Results

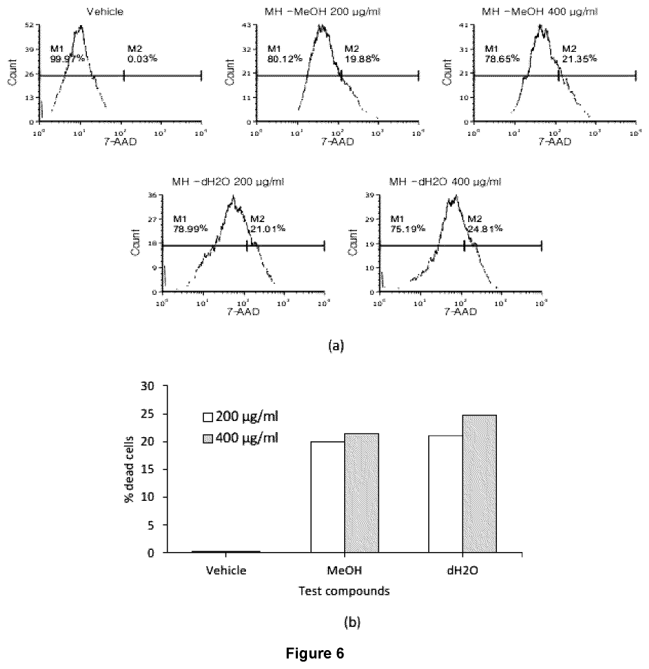

FIG. 6 shows the histograms and bar graphs displaying % dead cells. The preliminary results show approximately 20-25% cell death in MH extracts-treated cells in comparison to vehicle-treated cells. The histograms show that MH extracts have autofluorescence that may have interfered with the experimental data. Therefore, the findings are not consistent with the results obtained from MTS assay, where cell death measured in 7-AAD assay is markedly lower than cell death seen in MTS assay.

### Conclusion

Both the MTS assay of Example 1 and the 7-AAD assay of Example 2 show that methanol and water extracts of MH are cytotoxic to MDA-MB-231 cells at high treatment concentrations. Due to colour interference of extracts with certain fluorescence assays, an alternative viability assay, namely the acid phosphatase assay, is used in Example 3 to validate the degree of cytotoxicity of MH extracts on MDA-MB-231 cells.

### Example 3: Anticancer Effects of Chaga Extracted with Methanol Versus Water

### Extraction

For methanol extraction, 4 g of chaga powder was dissolved in 160 ml of methanol, sonicated twice for 20 min at room temperature, and centrifuged at 3000 rpm for 10 min. The supernatant was collected and evaporated using a rotatory evaporation system at 65° C. The concentrated extract was subjected to nitrogen evaporation to remove the solvent, and then dissolved in DMSO to a final concentration of 31 mg/ml.

For water extraction, 4 g of MH powder was dissolved in 160 ml water, sonicated twice for 20 min at room temperature, kept at room temperature for three days, vortexed, and centrifuged at 3000 rpm for 10 min. The supernatant was then freeze-dried into powder. The lyophilized powder was dissolved in DMSO to a final concentration of 31 mg/ml.

### MTS Assay

The potential inhibitory effects of chaga extracts on D-17 osteosarcoma cells (ATCC CCL 183) and WRL68 normal hepatocytes (ATCC CL-48™) were measured using an MTS assay (n=3). Briefly, 6000 cells were seeded and treated with different concentrations (100, 200, 400, 600, 800 μg/ml) of extracts for 24 h. Then, MTS/PMS reagent was added, and absorbance was measured at 490 nm using a plate reader.

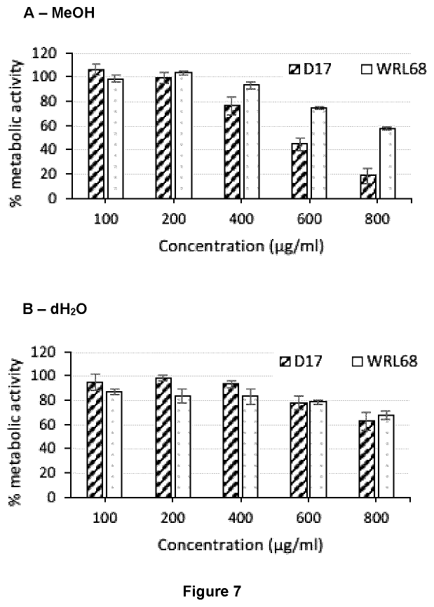

The MTS results (FIGS. 7A and 7B) show that the methanol extract of chaga markedly reduced the viability of D-17 osteosarcoma cells, while sparing WRL68 normal hepatocytes.

### Acid Phosphatase (ACP) Assay

Next, an acid phosphatase assay was carried out to validate the MTS assays results. This assay measures the metabolic activity of live cells in terms of cytosolic acid phosphatase activity by hydrolyzing the phosphatase substrate at acidic pH levels. Briefly, 6000 cells were seeded and treated with different concentrations (100, 200, 400, 600, 800 μg/ml) of extracts for 24 h. At the end of the incubation period, the plates were centrifuged at 400×g for 10 min, the supernatant was discarded, and cell monolayers were washed with PBS. Assay buffer (100 μL) (0.1 M sodium acetate; pH 5.5, 0.1% v/v Triton X-100 and 4 mg/mL phosphatase substrate) was added to each well and incubated for 2 hr at 37° C. 1N NaOH (50 μl) was added to each well and absorbance was measured at 405 nm using a plate reader.

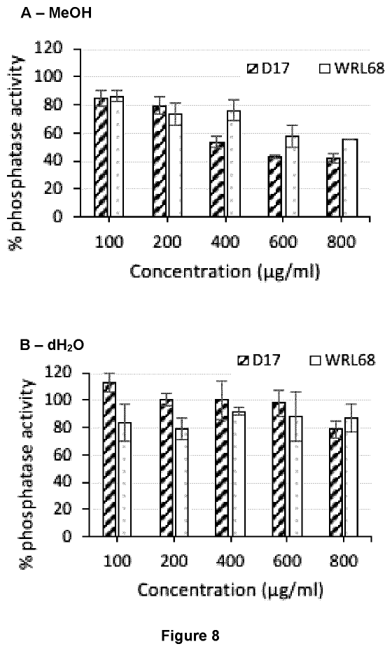

Like with the MTS assay, the ACP assay showed that the inhibitory effects of the methanol extract of chaga is markedly more cytotoxic in D-17 osteosarcoma cells than in WRL68 normal hepatocytes (FIGS. 8A and 8B).

### Morphology Assessment

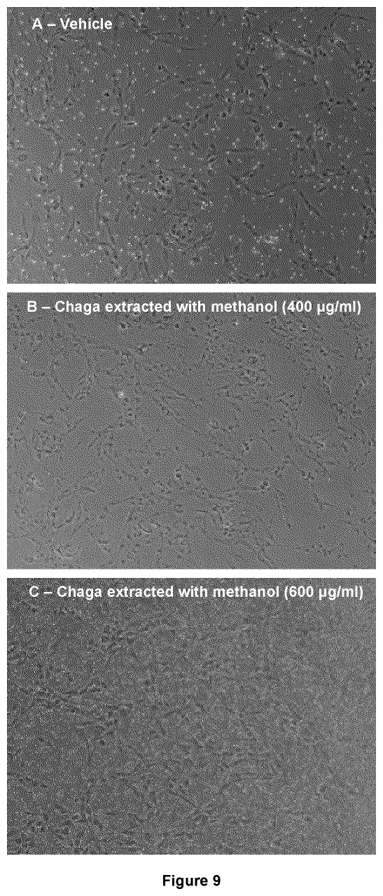

D-17 osteosarcoma cells were treated with vehicle or chaga extracted with methanol for 24 h at 37° C. The morphology of the cells was observed under an inverted phase contrast Nikon Eclipse E 100 microscope and images were captured at 100× magnification using an Infinity digital microscopy camera. FIGS. 9A, 9B, and 9C show a dose-dependent cytotoxicity to cancer cells of the extract.

### Example 4: Anticancer Effects of Phytoplankton Green Algae Chlorophyll-Containing Versus Chlorophyll-Free Fractions

### Extraction

From the results of Example 1, we hypothesized that chlorophyll might inhibit the cytotoxic activity of phytoplankton green algae extract. Therefore, here we attempted to isolate chlorophyll and compare the activity of chlorophyll-containing and chlorophyll-free fractions.

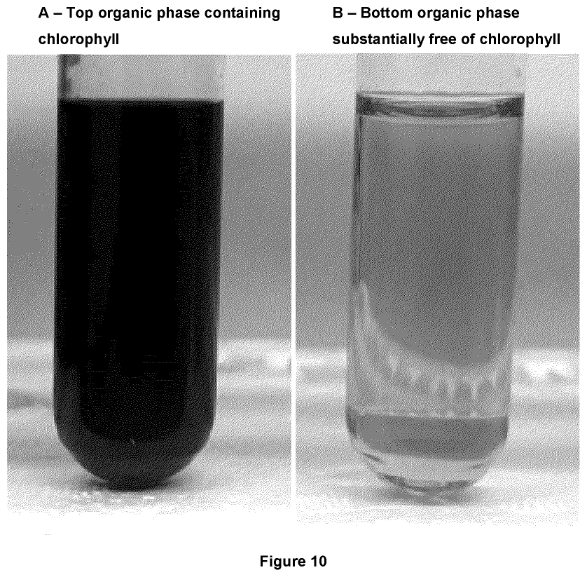

A 2-phase solvent system consisting of n-heptane/ethanol/acetonitrile/water (10:8:1:1, vol/vol) was used. Briefly, 3.5 mg of GL powder was mixed with respective amounts of solvent (total vol: 1.5 L), vigorously shaken and sonicated twice for 20 min at room temperature, and left to equilibrate at RT overnight in a separative funnel. Next day, the bottom aqueous phase was re-extracted using the same 2-phase solvent system (additional 1.2 L) to avoid incomplete separation due to saturation. The top phase (organic layer containing chlorophyll; FIG. 10A) and bottom phase (aqueous non-chlorophyll fraction; FIG. 10B) were separated into two containers. Both phases were gravity filtered, rotatory evaporated, nitrogen flushed, and then dissolved in DMSO to a final concentration of 31 mg/ml.

### MTS Assay

The potential inhibitory effects of phytoplankton green algae extracts on D-17 osteosarcoma cells (ATCC CCL 183) and WRL68 normal hepatocytes (ATCC CL-48™) were measured using an MTS assay (n=3). Briefly, 6000 cells were seeded and treated with different concentrations (100, 200, 400, 600, 800 μg/ml) of extracts for 24 h. Then, MTS/PMS reagent was added, and absorbance was measured at 490 nm using a plate reader.

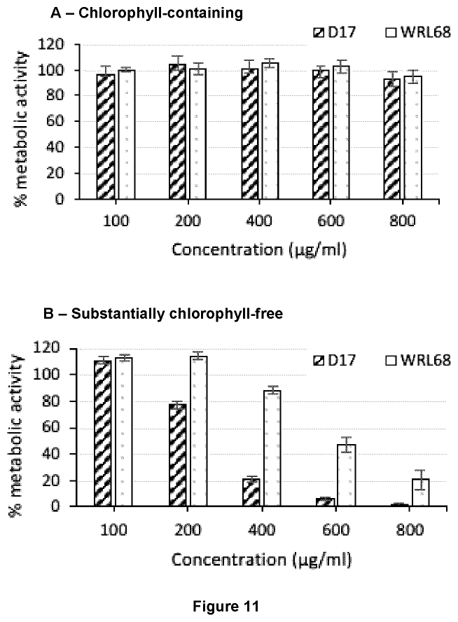

The MTS results (FIGS. 11A and 11B) show that the substantially chlorophyll-free of phytoplankton green algae markedly reduced the viability of D-17 osteosarcoma cells, while sparing WRL68 normal hepatocytes.

### Acid Phosphatase (ACP) Assay

Next, an acid phosphatase assay was carried out to validate the MTS assays results. This assay measures the metabolic activity of live cells in terms of cytosolic acid phosphatase activity by hydrolyzing the phosphatase substrate at acidic pH levels. Briefly, 6000 cells were seeded and treated with different concentrations (100, 200, 400, 600, 800 μg/ml) of extracts for 24 h. At the end of the incubation period, the plates were centrifuged at 400×g for 10 min, the supernatant was discarded, and cell monolayers were washed with PBS. Assay buffer (100 μL) (0.1 M sodium acetate; pH 5.5, 0.1% v/v Triton X-100 and 4 mg/mL phosphatase substrate) was added to each well and incubated for 2 hr at 37° C. 1N NaOH (50 μl) was added to each well and absorbance was measured at 405 nm using a plate reader.

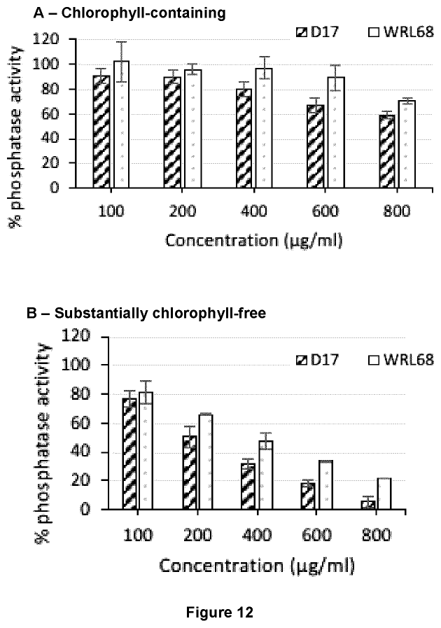

Like with the MTS assay, the ACP assay showed that the inhibitory effects of the substantially chlorophyll-free extract of phytoplankton green algae is markedly more cytotoxic in D-17 osteosarcoma cells than in WRL68 normal hepatocytes (FIGS. 12A and 12B).

### Morphology Assessment

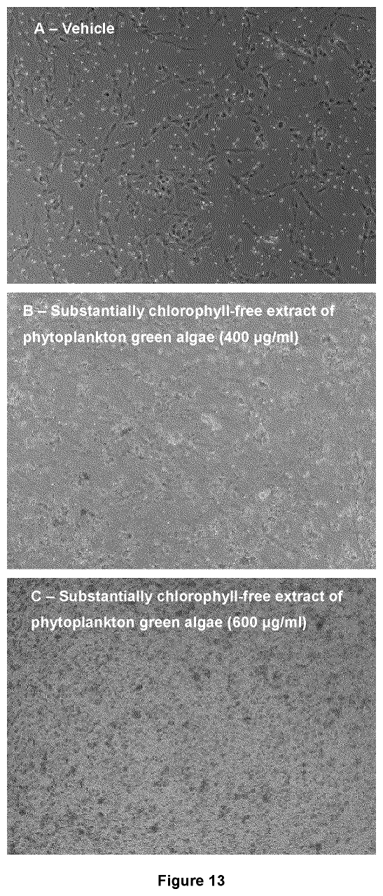

D-17 osteosarcoma cells were treated with vehicle or a substantially chlorophyll-free extract of phytoplankton green algae for 24 h at 37° C. The morphology of the cells was observed under an inverted phase contrast Nikon Eclipse E 100 microscope and images were captured at 100× magnification using an Infinity digital microscopy camera. FIGS. 13A, 13B, and 13C show a dose-dependent anticancer effect of the extract.

### Example 5: Natural Health Products to Manage Cancers of Dogs

### Background

Cancer is the major cause of death in adult dogs. For example, Canine osteosarcoma (OSA) is the most common form of canine bone neoplasia, where large and giant breeds are most at risk. The current standard course of treatment includes amputation, or limb-sparing surgery, followed by chemotherapy, most frequently using carboplatin, cisplatin, or doxorubicin. OSA is highly metastatic; when treated with amputation alone, dogs can face metastasis rates of up to 88%. Chemotherapy treatments increase the survival chances of dogs with OSA, and have some success in slowing, but not necessarily decreasing, the rate of metastasis. A drawback in the use of chemotherapeutic drugs is their toxicity, causing adverse effects in up to 48-76% of dogs. The negative effects caused by the toxicity of the current chemotherapeutic drugs and their inability to prevent metastasis illustrate the need for alternative approaches such as prevention and treatment by safe, less toxic, natural products. The objectives of this study were to (1) develop natural health products from Chaga wild mushroom (Inonotus obliquus) and phytoplankton (Tetraselmis chuffui); (2) determine the dose-depended cytotoxicity to selected five mammalian cancer cell lines: MCF-7 (ATCC-H2B-22, human breast carcinoma), HepG2 (ATCC-HB-8065, human liver carcinoma), HOS (ATCC-CRL-1543, human osteosarcoma), D-17 (ATCC-CCL-183, canine osteosarcoma), and DH-82 (ATCC-CRL-10389, canine histiocytic sarcoma), and (3) determine potential synergistic effects among most effective extracts.

### Materials and Methods

### Extracts Preparation

The natural product preparations from Chaga wild mushroom (MH) and phytoplankton microalgae (GL) were prepared as summarized in Table 1. The resulted yield are also presented in Table 1.

TABLE 1
The extracts of Chaga wild mushroom and phytoplankton
microalgae categories and their resulted yield.

| | | | Starting | | |
|---|---|---|---|---|---|
| | Sample | | quantity | Final yield | % |
| | code | Sample name | (g) | (mg) | Yield |
| | Chaga Wild Mushroom (MH) | | | | |
| 1 | MH-E1-SF | MH EtOH sugar-free | 30 | 804 | 2.68 |
| 2 | MH-SFE1b | 9000 psi, 50° C., 1h | 40 | 41 | 0.10 |
| 3 | MH-SFE1c | 7500 psi, 50° C., | 40 | 237 | 0.59 |
| | | 10% EtOH, 1h | | | |
| | Phytoplankton Microalgae (GL) | | | | |
| 4 | GLE1-CF | GL chlorophyll free | 2.5 | 270 | 10.8 |
| 5 | GLE1-CF- | GL chlorophyll free | 2.5 | 176.6 | 7.06 |
| | SF | and sugar-free | | | |

### Protocol of MH-E1-SF Preparation

1) Dissolve 30 g of MH in 600 mL of 100% EtOH.
2) Sonicate the sample for 20 min×2 at room temperature.
3) After sonication, centrifuge the sample at 3000 rpm for 10 min and filter through Fisher P8 filters (vacuum aided).
4) Collect the filtrate and re-extract bioactives from MH (pellets from centrifugation) using 300 mL of 100% EtOH.
5) Combine the filtrates from the first and second extractions.
6) Concentrate the collected filtrates down to 250 mL by using a rotovap system.
7) Equilibrate the flash chromatography column with 50% EtOH (in water) for 24 h.
8) Mix the concentrated sample with 250 mL of DI water and load it into the chromatography column.
9) Elute sugars in the loaded sample with DI water. Continue to elute with DI water until the Brix value of eluting falls below 0.1 (measure Brix value with handheld Brix meter).
10) Elute bioactives in the loaded sample by using 2 L of 100% EtOH.
11) Concentrate the EtOH elute by rotovaping and freeze-dry to generate a dry sample.

### Protocol for MH-SFE1b Preparation

The supercritical water extraction process consists of (i) a 500 mL/min dual piston pump, (ii) a 3 kW electric preheater (Diversified Metal Engineering Ltd. Charlottetown, PE, Canada), (iii) an 8 L stainless steel pressure vessel (Diversified Metal Engineering Ltd. Charlottetown, PE, Canada), and (iv) a shell and tube heat exchanger.

The operating conditions were 40 gram dried and finely ground Chaga powder at the pressure of 9000 psi, the temperature of 50° C., and the extraction time of 1 hour.

### Protocol for MH-SFE1c Preparation

The operating conditions were 40 gram dried and finely ground Chaga powder at the pressure of 7500 psi, the temperature of 50° C., cosolvent of 10% ethanol, and the extraction time of 1 hour.

### Protocol of GLE1-CF Preparation

1) Extraction with EtOH/water/acetonitrile/n-hexane (5.3:3:0.7:1). Solid: solvent ratio; 2.5 g of GL in 1 L of extraction solvent.
2) Phase separation in separation funnel to isolate chlorophyll into n-hexane phase.

Protocol;
1) Mix 2.5 g of GL with the first extraction solvent (530 of EtOH, 300 mL of DI water, and 70 mL of acetonitrile).
2) Sonicate the sample for 20 min×2 at room temperature.
3) After sonication, centrifuge the sample at 3000 rpm for 10 min and filter through Fisher P8 filters (vacuum aided).
5) Collect the filtrate into a separatory funnel and add 100 mL of n-hexane into the separatory funnel.
6) Shake the separatory funnel vigorously while degassing (opening the lid from time to time) to prevent pressure build-up.
7) Leave the separatory funnel undisturbed for 24 h to allow hexane and aqueous phases to separate. Note the green color hexane phase with chlorophyll on the top and yellowish aqueous phase on the bottom.
8) Collect the aqueous phase and concentrate by rotovaping.
9) Freeze dry the concentrated sample to generate a dry sample.

### Protocol of GLE1-CF-SF Preparation

1) Extraction with EtOH/water/acetonitrile/n-hexane (5.3:3:0.7:1). Solid: solvent ratio; 2.5 g of GL in 1 L of extraction solvent.
2) Phase separation in separation funnel to isolate chlorophyll into n-hexane phase.
3) Flash chromatography to generate a sugar-free sample.

Protocol;
1) Mix 2.5 g of GL with the first extraction solvent (530 of EtOH, 300 mL of DI water, and 70 mL of acetonitrile).
2) Sonicate the sample for 20 min×2 at room temperature.
3) After sonication, centrifuge the sample at 3000 rpm for 10 min and filter through Fisher P8 filters (vacuum aided).
4) Collect the filtrate into a separatory funnel and add 100 mL of n-hexane into the separatory funnel.
5) Shake the separatory funnel vigorously while degassing (opening the lid from time to time) to prevent pressure build-up.
6) Leave the separatory funnel undisturbed for 24 h to allow hexane and aqueous phases to separate. Note the green color hexane phase with chlorophyll on the top and yellowish aqueous phase on the bottom.
7) Collect the aqueous phase and concentrate by rotovaping.
8) Equilibrate the flash chromatography column with 50% EtOH (in water) for 24 h.
9) Mix the concentrated sample with 100% EtOH (1:1 v/v) and load it into the flash chromatography column.
10) Elute sugars in the loaded sample with DI water. Continue to elute with DI water until the Brix value of eluting falls below 0.1 (measure Brix value with handheld Brix meter).
11) Elute bioactives in the loaded sample by using 2 L of 100% EtOH.
12) Concentrate the EtOH elute by rotovaping and freeze-dry to generate a dry sample.

A stock solution of 25-50 mg/ml concentration was made in DMSO, filtered using 0.22 μm syringe filter, aliquoted, and stored at −20° C.

### Cell Lines, Culture Conditions and Reagents

The following five cancer cell lines were purchased from ATC through Cedarlane, Burlington, ON, Canada: MCF-7 (ATCC-H2B-22, Human breast Carcinoma), HepG2 (ATCC-HB-8065, Human liver carcinoma), HOS (ATCC-CRL-1543, Human Osteosarcoma), D-17 (ATCC-CCL-183, Canine osteosarcoma), and DH-82 (ATCC-CRL-10389, Canine Histiocytic Sarcoma). The cell lines were cultured in DMEM (Gibco) or EMEM (Sigma) supplemented with 10-15% FBS (Gibco) and 1% antibiotic in 5% CO₂ incubator at 37° C. All the experiments were performed after the second passage of the cells and repeated at least three times, independently.

Three extracts MH-E1-SF, MH-SFE1b, and MH-SFE1c, from Chaga wild mushroom and two extracts GLE1-CF and GLE1-CF-SF, prepared from the phytoplankton microalgae, were used for the preliminary screening of the cytotoxicity.

### Cell Viability Assay

IC₅₀ values of the Chaga wild mushroom and phytoplankton microalgae extracts in each cell line were confirmed with MTS colorimetric assay (Arumuggam et al., 2017). Briefly, the cells (5000-10000 cells/well) were seeded into 96-well plates and incubated overnight and treated with either DMSO control, 1, 50, 100, 300, or 500 μg/ml of each extract for 24 h and MTS/PMS reagent was added and incubated 2-3 h in the CO₂ incubator. Absorbance was measured at 490 nm (Infinite™ 200 series, Tecan, Switzerland). Background absorbance from the culture medium, DMSO, and extracts was subtracted to estimate the viability percentage. Graphpad Prism V 8.0 (GraphPad Software Inc., San Diego, CA, USA) was used to calculate IC₅₀ by sigmoidal dose-response curve.

Combination treatment and determination of Combination Index

With the IC₅₀ scores from the MTS viability assay, MH-E1-SF from the Chaga wild mushroom group and GLE1-CF-SF from phytoplankton microalgae group were selected for the determination of synergistic effects as both of the extracts showed strong significance in lower concentration when compared with the other extracts. A checkerboard assay with the concentration of 0, 25, 50, 100, 150, and 200 μg/ml for each test material in 96-well plate was assayed (Adil et al., 2019). The combination effect between two selected extracts was quantified using the method of isoboles (Tallarida, 20212; Huang et al., 2019). This procedure uses the IC₅₀ doses of individual drugs and uses these as intercept values in which doses are represented on x- and y-axes.

The isobole is expressed by the simple linear equation:

a/A+b/B=1

Where a is the dose of Drug A and b is the dose of drug B when the two drugs are used in combination.

Combination index (CI)<1 represents synergism, CI=1 represents additive effect and CI>1.5 represents antagonism.

### Statistical Analysis

Results are expressed as the mean±standard deviation (SD). Analysis of variance with post-hoc Tukey test was used for multiple comparisons using Graphpad Prism 8. A value of p<0.05 was considered statistically significant. *p<0.05, **p<0.01, ***p<0.001, ****p<0.0001.

### Results

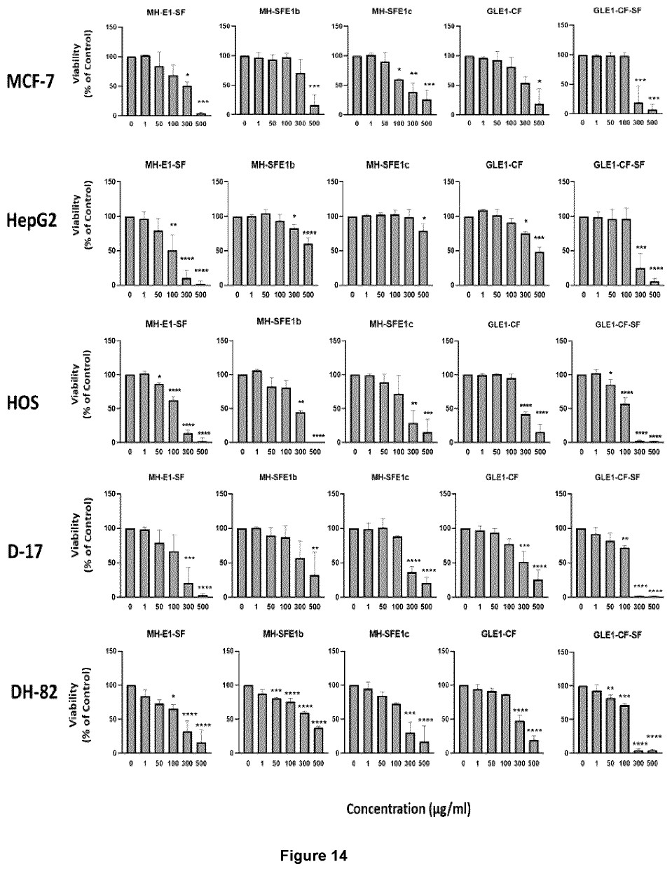

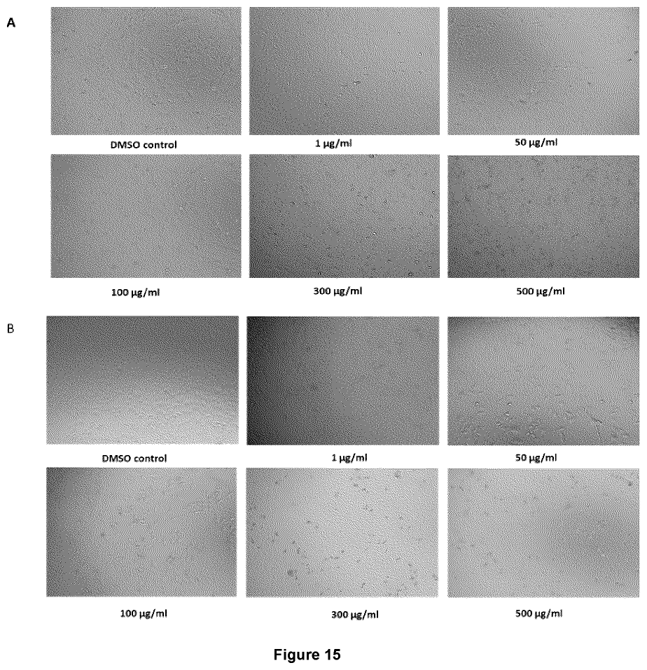

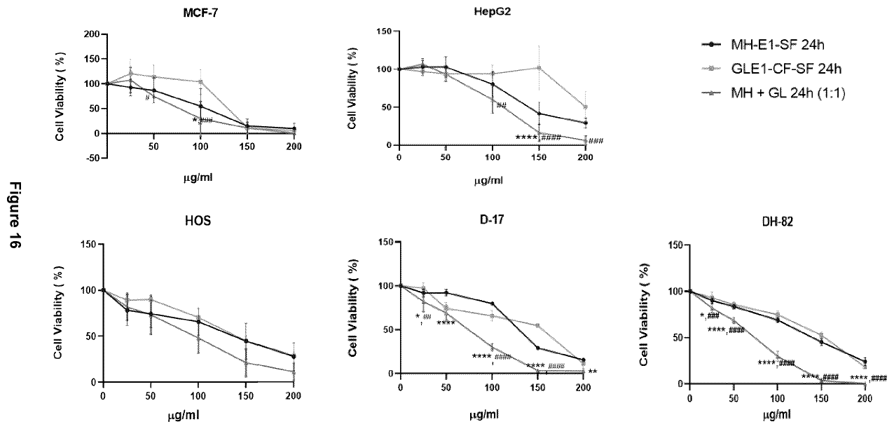

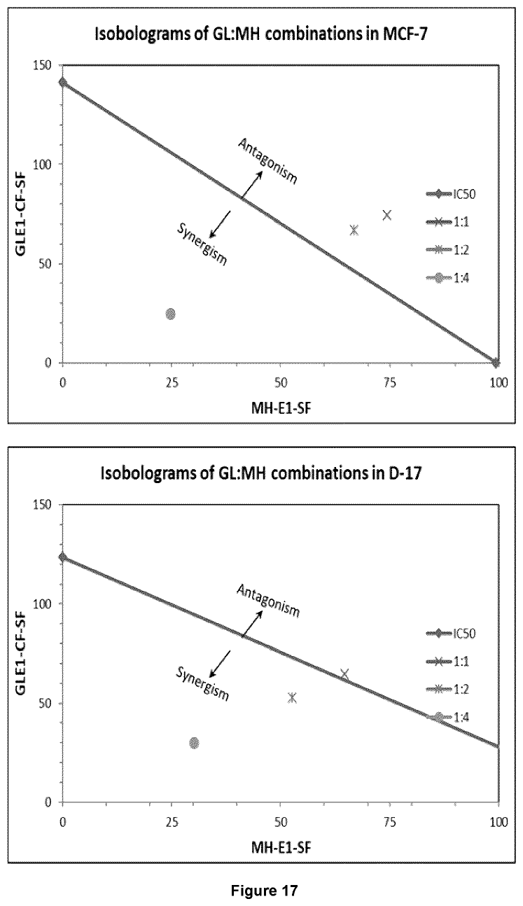

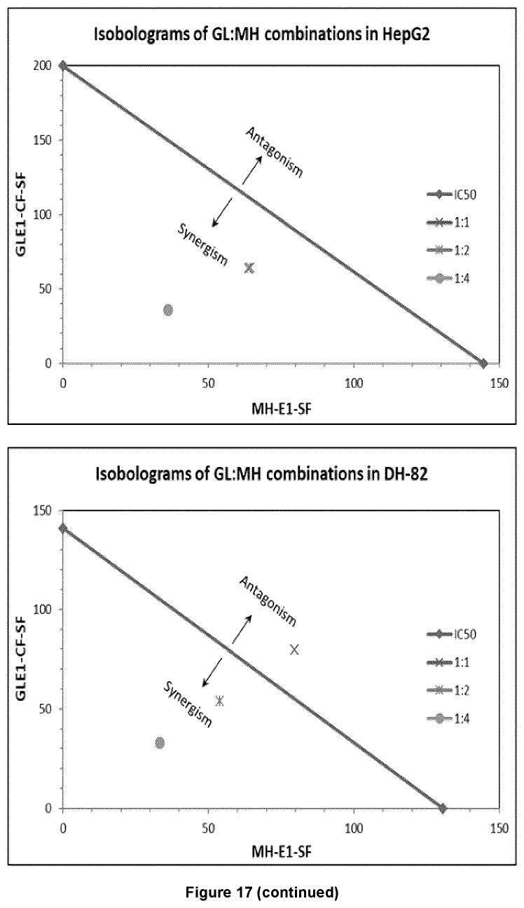

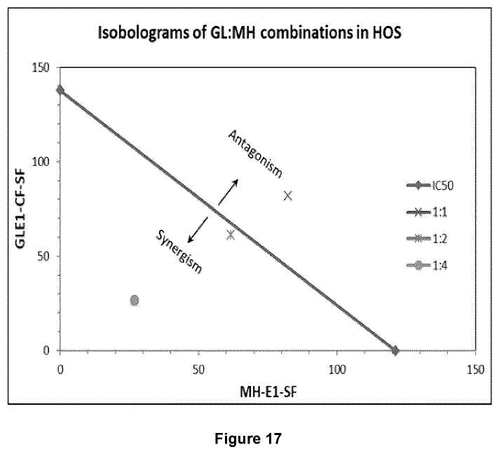

Human and canine cancer cell lines responded to the dose curve, and all the selected extracts inhibited the cell growth and viability at higher doses of 300 and 500 μg/ml (see FIGS. 14-16 and Table 2). In comparison to all the tested materials, MH-E1-SF and GLE1-CF-SF had the lowest IC₅₀ in all the tested cell lines, and therefore they were selected for the determination of the synergistic effect by various combinations (see FIG. 17 and Table 3).

TABLE 2
IC50 values of mushroom and algae extracts in various mammalian cancer cell lines.

| Cells/ | | | | | |
|---|---|---|---|---|---|
| Extracts | MH-E1-SF | MH-SFE1b | MH-SFE1c | GLE1-CF | GLE1-CF-SF |
| MCF7 | 186.7 ± 7.07 | 340.15 ± 23.82 | 181.76 ± 43.02 | 322.3 ± 37.90 | 198.65 ± 52.39 |
| HepG2 | 115.44 ± 31.62 | 559.25 ± 80.53 | 640 ± 173.24 | 501.35 ± 58.61 | 231.03 ± 13.159 |
| HOS | 124.9 ± 10.77 | 313.45 ± 21.00 | 261.95 ± 12.23 | 280.90 ± 15.08 | 107.62 ± 9.11 |
| D-17 | 172.4 ± 12.02 | 342.25 ± 52.11 | 235.4 ± 15.13 | 347.0 ± 53.17 | 131.06 ± 16.62 |
| DH-82 | 133.65 ± 29.34 | 357.5 ± 31.93 | 149.75 ± 9.40 | 246.1 ± 10.88 | 131.8 ± 1.980 |

TABLE 3
Combination index and isobole analysis of the various combination
of MH (MH-E1-SF) and GL (GL-E1-SF). Combination of GL and
MH (1:2 and 1:4) results in synergistic (bolded numbers)
and additive effect in the cancer cell lines. (CI <
1, synergy, CI > 1, additive and CI > 1.5, antagonist)

| Cells/ | GL:MH | GL:MH | MH:GL | GL:MH | MH:GL |
|---|---|---|---|---|---|
| Extracts | (1:1) | (1:2) | (1:2) | (1:4) | (1:4) |
| MCF7 | 1.274 | 1.145 | 1.145 | 0.424 | 0.413 |
| HepG2 | 0.76 | 0.76 | 0.778 | 0.43 | 0.407 |
| HOS | 1.274 | 0.95 | 0.999 | 0.415 | 0.455 |
| D-17 | 1.174 | 0.835 | 0.761 | 0.476 | 0.465 |
| DH-82 | 1.174 | 0.795 | 0.822 | 0.489 | 0.449 |

### Summary

Human and canine cancer cell lines responded to the drug dose curve of selected extracts and inhibited cell viability at the doses of 300 and 500 μg/ml. MH-E1-SF and GLE1-CF-SF had lower IC₅₀ and/or higher toxicity (significant) in the tested cell lines, and thus, they were selected for the determination of the synergistic effect by various combinations.

The summary of the observations for the individual cell lines is given below:

### MCF-7

The IC₅₀ of the extracts ranged from 181-340 μg/ml. The dose at 500 μg/ml of all the extracts inhibited the growth of MCF-7 cancer cells significantly, whereas MH-E1-SF, MH-SFE1c, GLE1-CF, and GLE1-CF-SF showed their cytotoxic properties at 300 μg/ml. The combination of MH-E1-SF and GLE1-CF-SF resulted in additive effect (1:1 and 1:2) and synergistic effects with 1:4 combination.

### HepG2

The IC₅₀ value for HepG2 cells ranged from 115-640 μg/ml. MH-SFE1b, MH-SFE1c, and GLE1-CF had higher IC₅₀ values 559, 640, and 501 μg/ml with high standard deviation as the extracts were effective only in 500 μg/ml dose. The combination of MH-E1-SF and GLE1-CF-SF resulted in a synergy effect with the 1:1, 1:2, and 1:4 combinations.

### HOS

The IC₅₀ value for human osteosarcoma ranged from 107-124 μg/ml. MH-E1-SF and GLE1-CF-SF showed their cytotoxic effect from 50 μg/ml. The combination of MH-E1-SF and GLE1-CF-SF resulted in additive effect (1:1) and synergistic with 1:2 and 1:4 ratios.

### D-17

The IC₅₀ value for canine osteosarcoma cells D-17 ranged from 131-347 μg/ml with higher doses strongly inhibiting the cells in all the extracts. The combination of MH-E1-SF and GLE1-CF-SF resulted in additive effect (1:1) and synergistic with 1:2 and 1:4 ratio.

### DH-82

The IC₅₀ value in histiocytic sarcoma cells DH-82 ranged from 131-357 μg/ml. 300 μg/ml of all the extracts significantly inhibited the growth with MH-E1-SF and GLE1-CF-SF showing cytotoxic effect at 100 μg/ml and 50 μg/ml, respectively. The combination of MH-E1-SF and GLE1-CF-SF resulted in additive effect (1:1) and synergistic with 1:2 and 1:4 ratio.

Overall, results from cell viability assay and the drug combination study suggest a therapeutic activity of Chaga wild mushroom and phytoplankton microalgae extracts in mammalian cancer cells.

### REFERENCES

Adil, M., Baig, M. H., & Rupasinghe, H. P. (2019). Impact of citral and phloretin, alone and in combination, on major virulence traits of Streptococcus pyogenes. Molecules, 24(23), 4237.
Arumuggam, N., Melong, N., Too, C. K., Berman, J. N., and Rupasinghe, H. P. V. (2017). Phloridzin docosahexaenoate, a novel flavonoid derivative, suppresses growth and induces apoptosis in T-cell acute lymphoblastic leukemia cells. American journal of cancer research, 7(12), 2452.
Tallarida, R. J. (2012). Revisiting the isobole and related quantitative methods for assessing drug synergism. Journal of Pharmacology and Experimental Therapeutics, 342(1), 2-8.
Huang, R. Y., Pei, L., Liu, Q., Chen, S., Dou, H., Shu, G., . . . and Fu, H. (2019). Isobologram analysis: A comprehensive review of methodology and current research. Frontiers in pharmacology, 10, 1222.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. An alcohol extract of chaga.

2. The alcohol extract of claim 1, wherein the alcohol extract is substantially carbohydrate-free.

3. The alcohol extract of claim 1 or 2, wherein the alcohol comprises a lower alcohol, such as methanol, ethanol, propanol, isopropanol, butanol, sec-butyl alcohol, isobutyl alcohol, t-butyl alcohol, or a combination thereof.

4. The alcohol extract of any one of claims 1 to 3, wherein the alcohol extract is collected from a supernatant of chaga dissolved in alcohol.

5. The alcohol extract of any one of claims 1 to 4, wherein the extract has anti-inflammatory effects.

6. The alcohol extract of any one of claims 1 to 5, wherein the extract has anti-oxidative effects.

7. The alcohol extract of any one of claims 1 to 6, wherein the extract stimulates the immune system.

8. The alcohol extract of any one of claims 1 to 7, wherein the extract has anticancer effects.

9. The alcohol extract of any one of claims 1 to 8, wherein the extract is preferentially cytotoxic to cancer cells as compared to non-cancerous cells.

10. The alcohol extract of any one of claims 1 to 9, further for preventing and/or treating inflammation.

11. The alcohol extract of any one of claims 1 to 10, further for preventing and/or treating oxidation.

12. The alcohol extract of any one of claims 1 to 11, further for stimulating the immune system.

13. The alcohol extract of any one of claims 1 to 12, for preventing and/or treating cancer.

14. The alcohol extract of claim 13, for preferentially killing cancer cells over non-cancerous cells.

15. The alcohol extract of any one of claims 1 to 14 for use in a veterinary animal such as a dog.

16. A substantially chlorophyll-free extract of phytoplankton green algae.

17. The substantially chlorophyll-free extract of claim 16, wherein the substantially chlorophyll-free extract is substantially carbohydrate-free.

18. The substantially chlorophyll-free extract of claim 16 or 17, wherein the phytoplankton green algae comprises microalgae.

19. The substantially chlorophyll-free extract of claim 18, wherein the microalgae comprises Spirulina, Chlorella, Tetraselmis, Nannochloropsis, Nitzchia, Navicula, Scenedesmus, Crypthecodinium, Chaetoceros, or combinations thereof.

20. The substantially chlorophyll-free extract of claim 19, wherein the microalgae comprises Nannochloropsis, such as Nannochloropsis gaditana, Tetraselmis, such as Tetraselmis chui, Chlorella, such as Chlorella vulgaris, or combinations thereof.

21. The substantially chlorophyll-free extract of claim 20, wherein the microalgae comprises Nannochloropsis gaditana, Tetraselmis chui, and Chlorella vulgaris.

22. The substantially chlorophyll-free extract of claim 21, wherein the microalgae comprises 60% w/w Nannochloropsis gaditana, 15% w/w Tetraselmis chui, and 25% w/w Chlorella vulgaris.

23. The substantially chlorophyll-free extract of any one of claims 16 to 22, wherein the substantially chlorophyll-free extract is collected from an aqueous phase of phytoplankton green algae dissolved in a 2-phase solvent.

24. The substantially chlorophyll-free extract of claim 23, wherein the 2-phase solvent comprises n-heptane, ethanol, acetonitrile and water.

25. The substantially chlorophyll-free extract of claim 24, wherein the n-heptane, ethanol, acetonitrile and water are in a ratio of 10:8:1:1, vol/vol, respectively.

26. The substantially chlorophyll-free extract of any one of claims 16 to 25, wherein the extract comprises less than about 5%, 4%, 3%, 2%, 1%, 0.5%, or 0.1% w/v chlorophyll.

27. The substantially chlorophyll-free extract of any one of claims 16 to 26, wherein the extract has anti-inflammatory effects.

28. The substantially chlorophyll-free extract of any one of claims 16 to 27, wherein the extract has anti-oxidative effects.

29. The substantially chlorophyll-free extract of any one of claims 16 to 28, wherein the extract stimulates the immune system.

30. The substantially chlorophyll-free extract of any one of claims 16 to 29, wherein the extract has anticancer effects.

31. The substantially chlorophyll-free extract of claim 30, wherein the extract is preferentially cytotoxic to cancer cells as compared to non-cancerous cells.

32. The substantially chlorophyll-free extract of any one of claims 16 to 31, for preventing and/or treating inflammation.

33. The substantially chlorophyll-free extract of any one of claims 16 to 32, for preventing and/or treating oxidation.

34. The substantially chlorophyll-free extract of any one of claims 16 to 33, for stimulating the immune system.

35. The substantially chlorophyll-free extract of any one of claims 16 to 34, for preventing and/or treating cancer.

36. The substantially chlorophyll-free extract of claim 35, for preferentially killing cancer cells over non-cancerous cells.

37. The substantially chlorophyll-free extract of any one of claims 16 to 36 for use in a veterinary animal such as a dog.

38. A combination comprising the alcohol extract of chaga of any one of claims 1 to 15 and the substantially chlorophyll-free extract of phytoplankton green algae of any one of claims 16 to 37.

39. The combination of claim 38, wherein the alcohol extract of chaga and the substantially chlorophyll-free extract of phytoplankton green algae are in synergistic amounts.

40. The combination of claim 39, wherein the synergistic amounts comprise a ratio of from about 1:10 to about 10:1 of the alcohol extract of chaga to the substantially chlorophyll-free extract of phytoplankton green algae.

41. The combination of claim 40, wherein the synergistic amounts comprise a ratio of from about 1:4 to about 4:1 of the alcohol extract of chaga to the substantially chlorophyll-free extract of phytoplankton green algae.

42. The combination of claim 41, wherein the synergistic amounts comprise a ratio of 1:4 or 4:1 of the alcohol extract of chaga to the substantially chlorophyll-free extract of phytoplankton green algae.

43. A composition comprising the combination of any one of claims 38 to 42.

44. A kit comprising the combination of any one of claims 38 to 42, optionally including instructions for use thereof.

45. A method for preventing and/or treating inflammation, the method comprising administering the methanol extract of any one of claims 1 to 15; the substantially chlorophyll-free extract of any one of claims 16 to 37; or the combination of any one of claims 38 to 42 to a subject in need thereof.

46. A method for preventing and/or treating oxidation, the method comprising administering the methanol extract of any one of claims 1 to 15; the substantially chlorophyll-free extract of any one of claims 16 to 37; or the combination of any one of claims 38 to 42 to a subject in need thereof.

47. A method for stimulating the immune system, the method comprising administering the methanol extract of any one of claims 1 to 15; the substantially chlorophyll-free extract of any one of claims 16 to 37; or the combination of any one of claims 38 to 42 to a subject in need thereof.

48. A method for preventing and/or treating cancer, the method comprising administering the methanol extract of any one of claims 1 to 15; the substantially chlorophyll-free extract of any one of claims 16 to 37; or the combination of any one of claims 38 to 42 to a subject in need thereof.

49. A method for preferentially killing cancer cells over non-cancerous cells, the method comprising administering the methanol extract of any one of claims 1 to 15; the substantially chlorophyll-free extract of any one of claims 16 to 37; or the combination of any one of claims 38 to 42 to a subject in need thereof.

50. The method of any one of claims 45 to 49, wherein the subject is a mammal.

51. The method of claim 50, wherein the mammal is a veterinary animal, such as a dog.

52. Use of the methanol extract of any one of claims 1 to 15; the substantially chlorophyll-free extract of any one of claims 16 to 37; or the combination of any one of claims 38 to 42 for preventing and/or treating inflammation in a subject.

53. Use of the methanol extract of any one of claims 1 to 15; the substantially chlorophyll-free extract of any one of claims 16 to 37; or the combination of any one of claims 38 to 42 for preventing and/or treating oxidation in a subject.

54. Use of the methanol extract of any one of claims 1 to 15; the substantially chlorophyll-free extract of any one of claims 16 to 37; or the combination of any one of claims 38 to 42 for stimulating the immune system in a subject.

55. Use of the methanol extract of any one of claims 1 to 15; the substantially chlorophyll-free extract of any one of claims 16 to 37; or the combination of any one of claims 38 to 42 for preventing and/or treating cancer in a subject.

56. Use of the methanol extract of any one of claims 1 to 15; the substantially chlorophyll-free extract of any one of claims 16 to 37; or the combination of any one of claims 38 to 42 for preferentially killing cancer cells over non-cancerous cells in a subject.

57. The use of any one of claims 52 to 57, wherein the subject is a mammal.

58. The use of claim 57, wherein the mammal is a veterinary animal, such as a dog.

59. A method for producing an active chaga extract, the method comprising dissolving the chaga in alcohol and collecting a resulting supernatant.

60. The method of claim 59, wherein the method further comprises sonicating the dissolved chaga before collecting the supernatant.

61. The method of claim 59 or 60, wherein the method further comprises centrifuging the dissolved chaga before collecting the supernatant.

62. The method of any one of claims 59 to 61, wherein the method further comprises evaporating the supernatant to remove methanol.

63. The method of claim 62, wherein the method further comprises dissolving the evaporated supernatant in a solvent.

64. A method for producing an active substantially chlorophyll-free phytoplankton green algae extract, the method comprising dissolving the phytoplankton green algae in a 2-phase solvent and collecting a resulting aqueous phase.

65. The method of claim 64, wherein the 2-phase solvent comprises n-heptane, ethanol, acetonitrile and water.

66. The method of claim 65, wherein the n-heptane, ethanol, acetonitrile and water are in a ratio of 10:8:1:1, vol/vol, respectively.

67. The method of any one of claims 64 to 66, wherein the method further comprises shaking and/or sonicating the dissolved phytoplankton green algae before collecting the collecting the aqueous phase.

68. The method of any one of claims 64 to 67, wherein the method further comprises equilibrating the dissolved phytoplankton green algae for a period of time before collecting the aqueous phase.

69. The method of any one of claims 64 to 68, wherein the method further comprises repeating the method by extracting the aqueous phase with the 2-phase solvent.

70. The method of any one of claims 64 to 69, wherein the method further comprises filtering the aqueous phase.

71. The method of any one of claims 64 to 70, wherein the method further comprises evaporating the aqueous phase.

72. The method of claim 71, wherein the method further comprises dissolving the evaporated aqueous phase in a solvent.

73. The method of any one of claims 64 to 72 wherein the extract comprises less than about 5%, 4%, 3%, 2%, 1%, 0.5%, or 0.1% w/v chlorophyll.

74. An extract made by the method of any one of claims 59 to 73.
